(19) United States
(12) Patent Application Publication
(10) Pub. No.: US 2004/0110272 A1
(43) Pub. Date: Jun. 10, 2004

(54) Biochemical vessel

(76) Inventor: Koji Fujita, Osaka (JP)

Correspondence Address: FULBRIGHT & JAWORSKI, LLP, 666 FIFTH AVE, NEW YORK, NY, 10103-3198, US

(21) Appl. No.: 10/722,976

(22) Filed: Nov. 26, 2003

(30) Foreign Application Priority Data
Dec. 10, 2002 (JP) 2002-358171

(51) Int. Cl. C12M 1/00

(52) U.S. Cl. 435/283.1

(57) ABSTRACT

A biochemical vessel has a plurality of sample holding cells juxtaposed one next to another. Each cell has a light transparent bottom. Each sample holding cell includes, in its inner side, a light reflecting face which extends radially away from the axis of the cell as the reflecting face extends downwards.

### BACKGROUND OF THE INVENTION

1. Field of the Invention

The present invention relates to a biochemical vessel having a plurality of sample holding cells juxtaposed one next to another, each cell having a light transparent bottom.

2. Description of the Related Art

For test or analysis of a sample such as a culture solution, it has been conventionally practiced to determine light or fluorescence emitting from the sample as a result of irradiation of ultraviolet rays or dripping of a chemical reagent thereto. The above-described biochemical vessel is designed to be set to a photometric or fluorometric device such as a microtiter tray reader, so that light such as light beam or fluorescence emitting from the sample held in the sample holding cell may be determined from under through the light transparent bottom.

In the case of the conventional biochemical vessel of the above-noted type, the inner face of each sample holding cell is formed as a cylindrical shape having a constant diameter (see e.g. Japanese Patent Application “Kokai” No. 2002-125656). In operation, the beam emitted downwards from the sample held in each sample holding cell can be determined from under through its light transparent bottom.

For this reason, for precision determination of even weak beam emitted from the sample, it was necessary to use an expensive biochemical vessel in which the bottom of its each sample holding cell is formed of a material having a particularly high light transparency. Hence, the determination would require high costs.

The present invention has been made in view of the above-described state of the art. The primary object of the invention is to enable high precision determination of even weak beam emitted from a sample without using such expensive biochemical vessel or photometric or fluorometric device.

### SUMMARY OF THE INVENTION

For accomplishing the above-noted object, according to a first characterizing feature of the present invention, there is provided a biochemical vessel having a plurality of sample holding cells juxtaposed one next to another, each cell having a light transparent bottom, wherein each sample holding cell includes, in its inner side, a light reflecting face which extends radially away from the axis of the cell as the reflecting face extends downwards.

With the above-described construction, the beam emitted upwards from the sample held in the sample holding cell will tend to impinge on the light reflecting face provided in the inner side and downwardly extending outwards in the radial direction of the sample holding cell and be reflected thereby toward the bottom of the sample holding cell. Accordingly, the amount of light to be transmitted through the bottom of the sample holding cell may be increased. As a result, precision determination is made readily possible even for weak beam emitted from the sample.

According to the second characterizing feature of the present invention, a portion or entirety of an inner peripheral face of the sample holding cell is formed as the light reflecting face which extends radially away from the axis of the cell as the reflecting face extends downwards.

This construction provides the advantage of eliminating need to provide separately the light reflecting face in the inner side of the sample holding cell. As a result, the construction of the vessel can be simple.

According to the third characterizing feature of the invention, the sample holding cell is formed by bonding one side of a plate-like member to a light transparent substrate, the plate-like member having a through hole whose diameter increases toward said one side thereof; and a portion or entirety of an inner peripheral face of the sample holding cell is formed as the light reflecting face which extends radially away from the axis of the cell as the reflecting face extends downwards.

With the above, it is easy to form, in the plate-like member, the through hole whose diameter increases toward the one side of the member. In addition, since the sample holding cell is formed by bonding this one side of the plate-like member to the light transparent substrate and a portion or entirety of an inner peripheral face of the sample holding cell is formed as the light reflecting face which extends radially away from the axis of the cell as the reflecting face extends downwards, it is possible to readily manufacture the biochemical vessel which allows precision determination even for weak beam emitted from the sample.

Further, since the sample holding cell has the progressively increasing diameter toward the lower portion thereof, that is, this sample holding cell has a narrowed mouth, when the vessel holds liquid sample therein, spilling of this liquid from the vessel can be restricted. Moreover, when the vessel stores a sample using a volatile solvent, volatilization of the solvent can be effectively restricted.

According to the fourth characterizing feature of the invention, the light reflecting face is formed as a mirror finished surface.

With the above, since the light reflecting face is formed as a mirror finished surface, the light impinged on the light reflecting face can be efficiently reflected toward the bottom of the sample holding cell, whereby the precision determination is further facilitated.

Further and other features and advantages of the invention will become apparent upon reading the following detailed description of preferred embodiments thereof with reference to the accompanying drawings.

### BRIEF DESCRIPTION OF THE DRAWINGS

FIG. 1 is a partially cutaway perspective view of a biochemical vessel,

FIG. 2 is an enlarged section view of principal portions,

FIG. 3 is an explanatory view of functions, and

FIG. 4 is an enlarged section view of principal portions showing a biochemical vessel relating to a second embodiment of the present invention.

### DESCRIPTION OF PREFERRED EMBODIMENTS

Preferred embodiments of the invention will be described with reference to the accompanying drawings.

[First Embodiment]

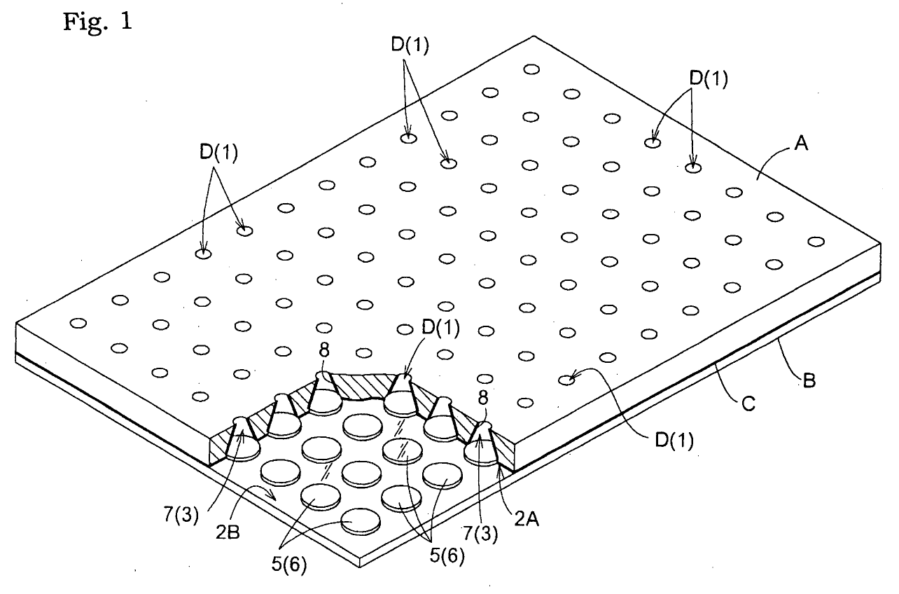

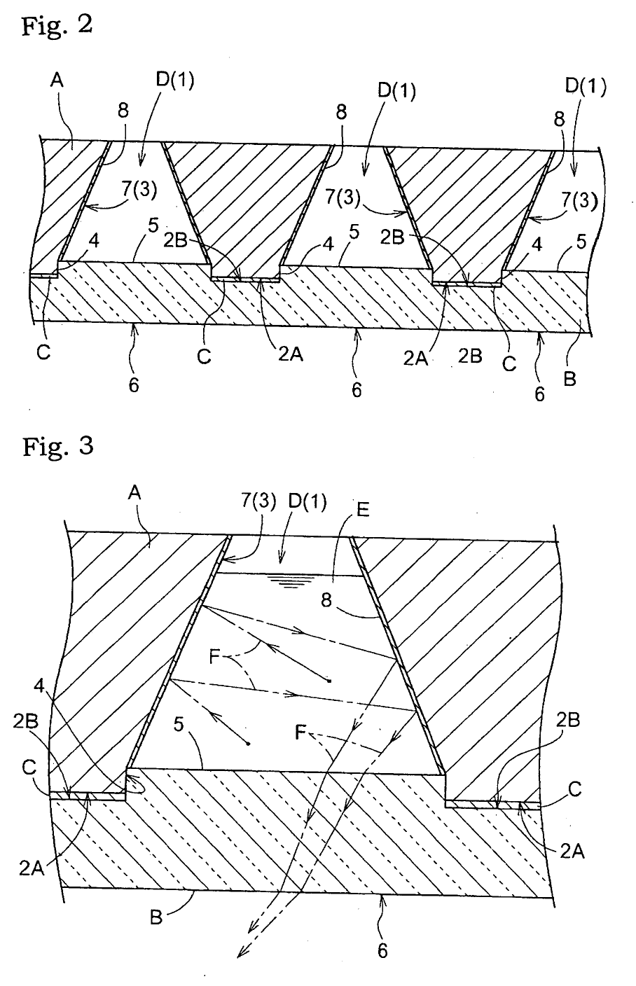

FIGS. 1 and 2 show a biochemical vessel having a plurality of sample holding cells D juxtaposed one next to another in the horizontal and vertical directions, each cell D having a light transparent bottom B. In forming this vessel, one side of a plate-like member A having a plurality of through holes 1 extending through the thickness of the member A is bonded to a rectangular glass substrate B (light transparent substrate) with an adhesive C, so that one end of each through hole 1 is covered with the glass substrate B, whereby the number of sample holding cells D are formed. Further, each sample holding cell D includes, in its inner side, a light reflecting face 7 which extends radially away from the axis of the cell as the reflecting face 7 extends downwards.

More particularly, as shown in details in FIG. 2, each through hole 1 includes a tapered inner peripheral face 3 having a truncated conical shape having a progressively increasing diameter to the one side of the bonded face 2A bonded to the glass substrate B and a cylindrical inner face 4 having a constant diameter and extending continuously from the large-diameter side end of the tapered inner peripheral face 3. A silver plated layer 8 is formed over the entire surface of the tapered inner peripheral face 3, so that the entire surface of the inner peripheral face of the sample holding cell D is provided as the mirror-surface light reflecting face 7 which extends radially away from the axis of the cell as the reflecting face 7 extends downwards.

Further, on the side of the bonded face 2B of the glass substrate B bonded to the plate-like member A, there are integrally formed a number of projections 5 in the form of cylindrical platforms having a substantially same outer diameter as the inner diameter of the cylindrical peripheral face 4 of the plate-like member A and having also a substantially same height as the length of the cylindrical inner peripheral face 4. Each of these projections 5 is fitted to the cylindrical inner peripheral face 4 along the entire circumference thereof. Then, the one side of the plate-like member A is bonded to the glass substrate B with the adhesive C. With these, there is formed the sample holding cell D having a progressively increasing diameter toward the lower portion thereof.

In operation, as shown in FIG. 3, beam F emitted upwards from a sample E such as a culture solution held within the sample holding cell D is reflected by the light reflecting face 7 provided inside the sample holding cell D, so that the beam can be readily guided toward the bottom 6 of the sample holding cell D. Therefore, with this construction, the amount of beam F transmitted through the bottom 6 of the sample holding cell D will be increased, compared with the conventional construction.

The plate-like member A can be formed of appropriate inorganic material such as various kinds of glass such as soda lime glass, various kinds of ceramics, various kinds of metal and this member A is formed with substantially same dimensions in its plan view as the glass substrate B. Alternatively, this plate-like member A can be formed also of various kinds of synthetic resin such as polystyrene, having UV transparency.

The glass substrate B can be formed of appropriate UV transparent glass having high UV transparency of 80% or more, hence suitable for ultraviolet spectrometry, including, but not limited to, natural quartz glass, synthetic quartz glass and borosilicate glass. Alternatively, the glass substrate B can be formed of UV transparent glass (Phillips' model: PH160) having an extremely high transparency of 85% or more for ultraviolet of 230 nm to 300 nm, hence particularly suitable for ultraviolet spectrometry.

The adhesive C comprises an inorganic adhesive such as low-melting glass, metal solder or the like. This is advantageous for avoiding e.g. elution of the adhesive C even when an organic solvent (e.g. isooctane) is held within the sample holding cell D for genetic analysis. However, the invention is not limited thereto and an organic adhesive may also be employed if appropriate.

[Second Embodiment]

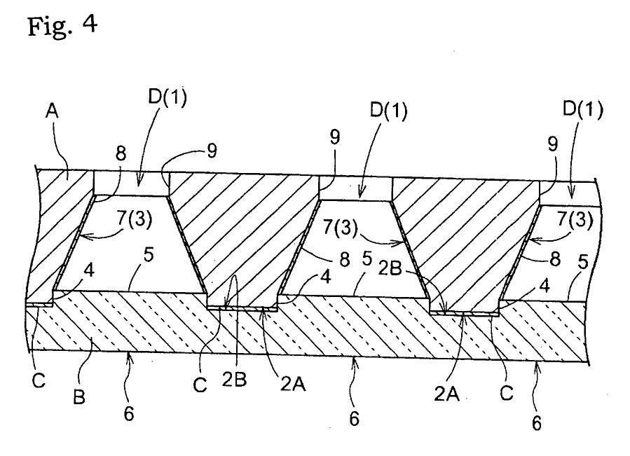

FIG. 4 shows a further embodiment wherein a portion of the inner peripheral face of the sample holding cell D is formed as a light reflecting face 7, provided as a mirror finished surface, which extends radially away from the axis of the cell as the reflecting face extends downwards. For forming this construction, a plurality of through holes 1 each having a cylindrical upper inner peripheral face 9 having a substantially constant diameter and extending continuously from the small-diameter side end of the tapered inner peripheral face 3 are formed in the plate-like member A. Then, the glass substrate B is bonded to this plate-like member A, thereby to form a plurality of sample holding cells D having a progressively increasing diameter toward the lower portion thereof. Further, a silver plated layer 8 is formed over the substantially entire surface of the tapered inner peripheral face 3, so that a portion of the inner peripheral face of the sample holding cell D is provided as the mirror-surface light reflecting face 7 which extends radially away from the axis of the cell as the reflecting face extends downwards.

The rest of the construction is identical to that of the first embodiment described above.

[Other Embodiments]

1. With the biochemical vessel of the invention, the light reflecting face which extends radially away from the axis of the cell as the reflecting face extends downwards may be provided separately in the inner side of the sample holding cell.

2. With the biochemical vessel of the invention, the inner peripheral face of the sample holding cell can be formed in a partial spherical shape to form the light reflecting face which extends radially away from the axis of the cell as the reflecting face extends downwards.

3. With the biochemical vessel of the invention the vessel may include a flat light reflecting face which extends radially away from the axis of the cell as the reflecting face extends downwards.

4. With the biochemical vessel of the invention, the vessel can be constructed such that the beam emitted upwards from the sample held within the sample holding cell may be totally reflected by the light reflecting face which extends radially away from the axis of the cell as the reflecting face extends downwards.

In these manners, the invention may be embodied in any other manner as described above. Further changes or modifications will be apparent for those skilled in the art from the foregoing disclosure within the scope of the invention defined in the appended claims.

1. A biochemical vessel having a plurality of sample holding cells juxtaposed one next to another, each cell having a light transparent bottom, wherein each sample holding cell includes, in its inner side, a light reflecting face which extends radially away from the axis of the cell as the reflecting face extends downwards.

2. The biochemical vessel according to claim 1, wherein a portion or entirety of an inner peripheral face of the sample holding cell is formed as the light reflecting face which extends radially away from the axis of the cell as the reflecting face extends downwards.

3. The biochemical vessel according to claim 2, wherein the sample holding cell is formed by bonding one side of a plate-like member to a light transparent substrate, the plate-like member having a through hole whose diameter increases toward said one side thereof; and a portion or entirety of an inner peripheral face of the sample holding cell is formed as the light reflecting face which extends radially away from the axis of the cell as the reflecting face extends downwards.

4. The biochemical vessel according to any one of claims 1-3, wherein the light reflecting face is formed as a mirror finished surface.
